(19) United States
(12) Patent Application Publication
(10) Pub. No.: US 2011/0172644 A1
(43) Pub. Date: Jul. 14, 2011

(54) MULTI LAYER COEXTRUDED CATHETER SHAFT

(76) Inventors: Michael S. Zanoni, Glens Falls, NY (US); Thomas V. Casey, II, Troy, NY (US); Brian F. Nentwick, Greenfield Center, NY (US); Daniel K. Recinella, Queensbury, NY (US); William M. Appling, Granville, NY (US)

(21) Appl. No.: 13/071,660

(22) Filed: Mar. 25, 2011

Related U.S. Application Data
(63) Continuation-in-part of application No. 12/566,141, filed on Sep. 24, 2009.
Application No. 12/566,141 is a continuation of application No. 10/728,267, filed on Dec. 4, 2003, now Pat. No. 7,618,411.
(60) Provisional application No. 60/430,998, filed on Dec. 4, 2002.

(51) Int. Cl. A61M 25/098

(52) U.S. Cl. 604/529

(57) ABSTRACT

A central venous catheter is provided having an outer tubular member and an inner tubular member that are preferably formed as a single integrated tube containing polymer material of different durometer and varying amounts of radiopaque filler material. The polymer durometer of the inner tubular member is higher than the polymer durometer of the outer tubular member. The percentage by weight of the filler material contained in the inner tubular member is higher than that of the outer tubular member. The combination of the higher durometer inner tubular member and the lower durometer outer tubular member along the length of the tube provides the desired tensile strength, hardness, chemical resistance and fatigue resistance and at the same time provides the desired flexibility and radiopacity.

### CROSS REFERENCE TO RELATED APPLICATIONS

This application is a continuation-in-part application of U.S. application Ser. No. 12/566,141, filed Sep. 24, 2009, which is a continuation application of U.S. application Ser. No. 10/728,267, filed Dec. 4, 2003, which application claims priority to U.S. Provisional Application Ser. No. 60/430,998, filed Dec. 4, 2002, which applications are incorporated herein by reference in their entireties.

### FIELD OF THE INVENTION

The present invention relates to a medical device apparatus and method for the delivery and withdrawal of fluids and medications. More particularly, the present invention relates to a venous access catheter device with shaft characteristics for enhanced catheter performance and method of manufacture.

### BACKGROUND

Venous access catheters provide venous access to the central circulatory system. Venous access catheters include central venous catheters, dialysis catheters and peripherally inserted central catheters, also known as PICC lines. The access line is used for the delivery of intravenous fluids, medications such as chemotherapy drugs and antibiotics, and blood products. Venous access catheters may also be used as access mechanisms for blood sampling and the administration of contrast agents during diagnostic Computer Tomography (CT) procedures.

One type of venous access catheters, PICC lines, provide venous access to the central circulatory system through a peripheral vein. PICC lines have been in use for many years with a variety of configurations. These include single lumen, dual lumen and other multi-lumen configurations. They come in various lengths to accommodate different anatomy and catheter insertion sites. Generally, a PICC line is inserted through a peripheral location such as the arm, with the tip placed in the central circulation, such as the superior vena cava. The PICC line is designed to remain within the patient for a period of one week to a year and can be accessed in an inpatient, outpatient or home setting.

The majority of the PICC lines presently on the market are made from single material such as silicone rubber or polyurethane. While these catheters are biocompatible and designed to minimize indwelling side effects and optimize patient comfort, they do have several drawbacks. The soft material characteristics of the catheter provide patient comfort but increase insertion difficulties and reduce the long-term durability of the catheter. The material characteristics of the catheter shaft also restrict use to only low pressure injections, typically less than 100 psi.

A PICC line is typically inserted percutaneously, either under fluoroscopic guidance or using a bedside, “blind” approach followed by x-ray imaging to confirm correct tip placement within the vessel. With either technique, the medical professional must confirm that the distal tip of the PICC line is located within the superior vena cava, rather than in the jugular vein or other unintended vessel. Typically, a post-placement x-ray is used to visualize the distal segment of the catheter within the body. Most venous access catheters do not have sufficient radiopacity to allow for easy visualization of the distal tip.

Typically, a PICC line should be sufficiently flexible so that it minimizes patient discomfort and does not cause trauma to the vein wall during insertion or over prolonged periods while still being sufficiently rigid enough to facilitate insertion over a guidewire, i.e., pushability and resistance to kinking during and after insertion require a stiffer shaft material. These opposing technical requirements have been partially addressed by some manufacturers by incorporating a softer tip welded to the catheter shaft. While this design provides a soft, atraumatic distal end allowing a stiffer, more rigid shaft body, the catheter is uncomfortable to the patient because the majority of the shaft is stiff. In addition, the physician cannot customize the length of these catheters by cutting at the tip, as is commonly done in the practice. In some cases, PICCs have problems with guidewire sticking.

Another problem with most conventional venous access catheters is that they do not have sufficient radiopacity to allow for easy visualization of the distal tip of the catheter. Attempts to address this problem have involved providing a highly radiopaque distal tip bonded or otherwise welded to the catheter shaft, which can decrease the overall strength of the catheter and increase the risk of fracture at the bond or weld point. Thus, in addition to this design being uncomfortable to the patient because the majority of the shaft is stiff, a physician cannot selectively customize the length of these catheters by cutting at the radiopaque tip, as is commonly done in practice.

Other catheter designs have attempted to provide acceptable distal radiopacity levels by using highly filled polymer throughout the entire shaft length. Although providing an acceptable level of visibility, the highly filled shaft material can have poor fatigue and chemical resistance, which can result in an increased occurrence of shaft fracture due to external exposure conditions. The resulting catheter shaft can be subject to failure at the proximal end where the catheter exits the body. At the point at which the catheter shaft exits the patient's body, the catheter is exposed to extensive bending, manipulation, and surface contact with site care chemicals such as antibiotics and antiseptics.

Some PICC line designs include a separate obturator or other type stiffener device to provide additional stiffness during insertion. Once inserted and positioned, the obturator is removed from the lumen of the PICC line. While this design has the advantage of ease of insertion, the catheter shaft is typically soft and not radially strong enough to handle the internal pressures associated with CT injections. In addition, for multi-lumen PICC lines, the medical professional must be cognizant of which lumen to insert the obturator into as incorrect insertion may damage the catheter.

Most conventional PICC lines have a capability of withstanding less than 100 pounds per square inch (psi). This is particularly true of silicone-based PICC lines. Although most PICC line pressure capabilities are sufficient for the delivery of medications and for sampling of blood, they are not designed for delivery of contrast media using a power injector. In one aspect, power injectors are used in radiology suites as a method for rapidly delivering diagnostic contrast media, particularly for CT applications. Contrast media delivered using a power injector can reach high injection pressures, for example, up to about 300 psi. Although an in-place PICC line provides an available delivery path for the contrast media, it often cannot be used because the PICC line cannot withstand the higher pressures generated when using a power injector. Instead, the physician must access the patient's vein in another location using a short IV-type catheter designed to withstand higher pressures.

Patients with PICC lines are often very ill and gaining access to a vein is difficult for the caregiver on the one hand while it is as painful and traumatic for the patient on the other hand. Continuous access of the venous system by IV needles or catheters results in eventual destruction of the available veins. Accordingly, being able to access the venous system using an already-in-place PICC line would have significant advantages to both the patient and the health care providers.

Therefore, it is desirable to provide a variable-characteristic venous access catheter that is sufficiently rigid for ease of placement and yet sufficiently flexible so as not to damage vessels.

It is also desirable to provide a venous access catheter that is comfortable to the patient and also has sufficient durability including chemical and fatigue resistance to withstand prolonged indwelling times.

It is further desirable to provide a venous access catheter that can withstand higher-pressure injections generated by power infusion devices without causing catheter damage.

It is also desirable to provide a venous access catheter that is designed as a one-piece construction for enhanced reliability and strength.

It is further desirable to provide a venous access catheter with a distal segment having enhanced visibility under x-ray or fluoroscopy to aid in placement of the catheter without compromising overall catheter strength.

It is further desirable to provide a venous access catheter having a dual-layer coaxial configuration that provides enhanced flexibility and increased strength.

Various other purposes and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description.

### SUMMARY

A central venous catheter is provided. This central venous catheter has catheter shaft having an outer tubular member and an inner tubular member, each having an inner and an outer surface. The inner tubular member is coaxially disposed within the outer tubular member, together defining a total catheter wall thickness. The outer tubular member is comprised of a first polymer material having a first durometer and a first amount of radiopaque filler. The inner tubular member is comprised of a second polymer material having a second durometer and a second amount of radiopaque filler. The second durometer is greater than the first durometer, and the second amount of radiopaque filler is greater than the first amount of radiopaque filler.

A central venous catheter having a proximal tube segment, a distal tube segment and a transition tube segment interposed between the proximal and distal tube segments is provided. The three segments are preferably formed as a single integrated tube containing polymer material of different durometer and different amounts of radiopaque filler material. The polymer durometer of the proximal segment is higher than the polymer durometer of the distal segment. By contrast, the percentage by weight of the filler material contained in the distal segment is higher than that of the proximal segment. The variation in the polymer durometer and the filler amount along the length of the tube provides the desired tensile strength, hardness, chemical resistance and fatigue resistance at the proximal segment and at the same time provides the desired flexibility and radiopacity at the distal segment.

### BRIEF DESCRIPTION OF THE DRAWINGS

FIG. 1 is a plan view of a venous access catheter.

FIG. 2 is a plan view of the venous access catheter of FIG. 1 which has been inserted into a patient and enlarged partial plan views of the proximal and distal segments of the catheter.

FIG. 3A is a graph depicting one method of altering the filler amount and durometer levels of the polymer material to achieve the variable characteristics of the venous access catheter.

FIG. 3B is a table listing the test results of the filler and durometer mixture of FIG. 3A.

FIG. 4 is a plan view of an alternative embodiment of a venous access catheter.

FIG. 5 is a cross-sectional view of the venous access catheter of FIG. 4.

### DETAILED DESCRIPTION OF THE INVENTION

The present invention can be understood more readily by reference to the following detailed description and the examples included therein and to the Figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges can be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, the words “proximal” and “distal” refer to directions away from and closer to, respectively, the insertion tip of the catheter described herein. The terminology includes the words above specifically mentioned, derivatives thereof, and words of similar import.

Other than in the operating examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values can be used.

“Formed from” and “formed of” denote open claim language. As such, it is intended that a member “formed from” or “formed of” a list of recited components and/or materials be a member comprising at least these recited components and/or materials, and can further include other non-recited components and/or materials.

Examples provided herein, including those following “such as” and “e.g.,” are considered as illustrative only of various aspects and features of the present disclosure and embodiments thereof, without limiting the scope of any of the referenced terms or phrases either within the context or outside the context of such descriptions. Any suitable equivalents, alternatives, and modifications thereof (including materials, substances, constructions, compositions, formulations, means, methods, conditions, etc.) known and/or available to one skilled in the art can be used or carried out in place of or in combination with those disclosed herein, and are considered to fall within the scope of the present disclosure. Throughout the present disclosure in its entirety, any and all of the one, two, or more features and aspects disclosed herein, explicitly or implicitly, following terms “example”, “examples”, “such as”, “e.g.”, and the likes thereof may be practiced in any combinations of two, three, or more thereof (including their equivalents, alternatives, and modifications), whenever and wherever appropriate as understood by one of ordinary skill in the art. Some of these examples are themselves sufficient for practice singly (including their equivalents, alternatives, and modifications) without being combined with any other features, as understood by one of ordinary skill in the art. Therefore, specific details disclosed herein are not to be interpreted as limiting, but merely as a basis for the claims and as a representative basis for teaching one skilled in the art to variously employ aspects and features of the present disclosure in virtually any appropriate manner.

“Polymer” or “polymeric” refers to a natural, recombinant, synthetic, or semisynthetic molecule having in at least one main chain, branch, or ring structure having two or more repeating monomer units. Polymers broadly include dimers, trimers, tetramers, oligomers, higher molecular weight polymers, adducts, homopolymers, random copolymers, pseudocopolymers, statistical copolymers, alternating copolymers, periodic copolymers, bipolymers, terpolymers, quaterpolymers, other forms of copolymers, substituted derivatives thereof, and mixtures thereof, and narrowly refer to molecules having one or more repeating monomer units. Polymers can be linear, branched, block, graft, monodisperse, polydisperse, regular, irregular, tactic, isotactic, syndiotactic, stereoregular, atactic, stereoblock, single-strand, double-strand, star, comb, dendritic, and/or ionomeric, can be ionic or non-ionic, can be neutral, positively charged, negatively charged, or zwitterionic, and can be used singly or in combination of two or more thereof.

As used herein, “substantially”, “generally”, and other words of degree are relative modifiers intended to indicate permissible variation from the characteristic so modified. It is not intended to be limited to the absolute value or characteristic which it modifies, but rather possessing more of the physical or functional characteristic than its opposite, and preferably, approaching or approximating such a physical or functional characteristic. “Optional” or “optionally” means that the subsequently described element, event or circumstance can or cannot occur, and that the description includes instances where said element, event or circumstance occurs and instances where it does not.

Referring now in detail to the drawings, in which like reference numerals indicate like parts or elements throughout the several views, in various embodiments, presented herein is an exemplary catheter shaft and method of manufacture.

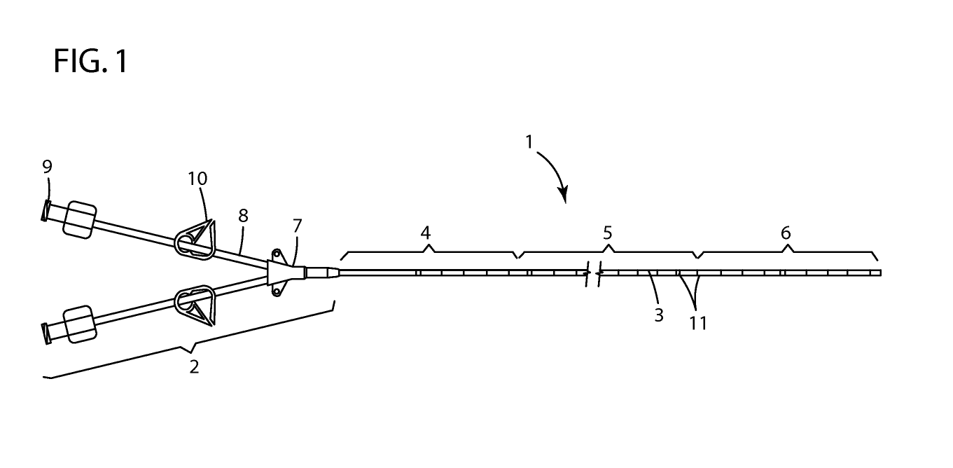

Referring to FIG. 1, a variable characteristic PICC line is shown from a plan view. In one aspect, the catheter 1 is comprised of a hub section 2, a tube or shaft 3 with a substantially rigid proximal segment 4, a transition segment 5 and a substantially flexible distal segment 6. In the embodiment shown, a dual-lumen catheter is provided. In this embodiment, the hub 2 is further comprised of a bifurcated hub component 7 and two extension legs 8 corresponding to each shaft lumen, as is well known in the art. The extension legs 8 terminate at the proximal end with a connector such as a standard luer fitting 9 for connection to injection or aspiration devices. Leg clamps 10 coaxially arranged around the extension legs 8 may be used to clamp off or occlude the leg lumens, thereby selectively preventing the inflow or outflow of fluids through the catheter 1. The catheter may include measurement markers 11 to assist in placement within the vessel.

In one exemplary aspect, a unitary or otherwise monolithic, variable characteristic catheter shaft for a central venous catheter such as a PICC line is provided. It is contemplated that the characteristics can optionally comprise varying flexibility along the shaft, increased radiopacity at the distal segment 6 and/or enhanced tensile strength and durability at the proximal segment 4.

In one aspect, the shaft, at the proximal segment 4, can be formed to be stiffer and stronger than the distal segment 6. The transition segment 5, which is interposed between the respective proximal and distal segments 4, 6 can be configured such that it has more flexibility than the distal segment 6. In one aspect, it is contemplated that, within transition segment 5, the flexibility may vary from less flexible at the proximal end of the transition segment to more flexible at the distal end of the transition segment. The distal segment 6 can be more flexible than transition segment 5 and substantially more flexible than proximal segment 4.

The variable flexibility characteristics of the present catheter 1 provide important advantages over conventional PICC lines. In one aspect, the relative increased rigidity and columnar strength of the proximal segment 4 of the catheter shaft 3 provides the user with increased pushability and control during insertion and advancement through the vessel. Thus, in operation, the increased stiffness of the proximal segment 4 relative to the distal segment 6 allows for the PICC line to be inserted and advanced easily with or without the use of a conventional guidewire.

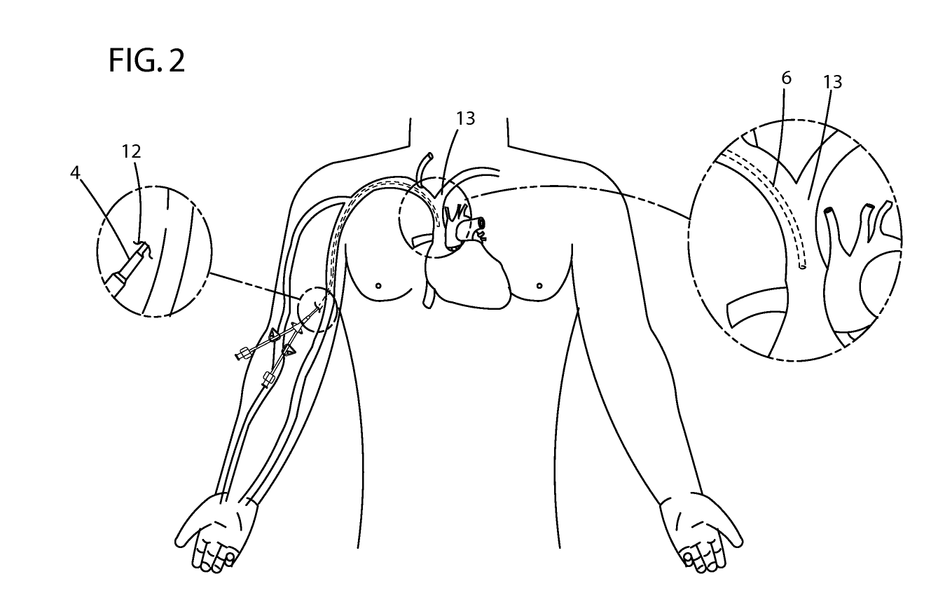

In a further aspect, the distal segment 6 also provides advantages over traditional PICC lines. The flexible soft shaft can be configured at the distal segment 6 to be substantially similar to the flexibility of silicone catheters. In this aspect, the shaft can minimize vessel wall trauma caused from contact with the shaft, particularly along distal segment 6 as shown in FIG. 2. Vessel trauma has been shown to increase the risk of thrombus formation, with its resulting complications including catheter occlusion. Thus, decreasing vessel wall trauma over the extended implantation time may contribute to a lower risk of thrombus formation, catheter occlusion and other procedural complications.

In addition to the variable flexibility along the shaft length described above, it is contemplated that the PICC line can be configured to enhanced durability and tensile strength at the proximal segment 4 of the shaft. In another aspect, the proximal portion 4 can also provide for increased strength and durability for that portion of the catheter shaft that is exposed outside of the patient, as shown in FIG. 2. The risks of damage from patient movement, stress at the insertion site 12, and decreased shaft integrity from long-term exposure to chemical substances used during medical procedures are all minimized by providing a PICC line having a stiffer, stronger proximal section 4.

Although implanted PICC lines provide access to the vasculature for administration of fluids, conventional PICC lines typically cannot be used for CT power injections because the shaft cannot withstand the internal pressures generated during the injection, which may be as high as 300 pounds per square inch (psi). Conventionally, CT injections can be administered as part of a diagnostic imaging procedure to determine the presence or status of a disease state. In operation, a CT power injector is connected to a high-pressure fluid line and then to an access needle. The injections are delivered over a period of time at a desired flow rate. Typically, contrast media is delivered through an IV needle or catheter at a rate of approximately 2-4 cc per second, with a total delivered volume of between 150 and 200 mls. Although venous access is available through the PICC line, conventional catheters with their relatively low burst strength cannot withstand the prolonged pressure generated during the CT injections. Commonly, PICC lines are accompanied by warnings advising against high-pressure conditions over 100 psi, making them un-usable for the delivery of contrast media during diagnostic imaging procedures.

As a result of the limitations of conventional PICC lines, a physician needs to gain separate access with an IV-type needle. Typically, a needle is placed in the forearm area and is used to inject contrast media during the diagnostic CT procedure. This separate access site increases the complexity and time of the diagnostic procedure in addition to increasing the risks associated with a second access site such as bleeding, hemotomas and infection.

With the disclosed catheter shaft 3, however, CT injections through the PICC line are possible without the risk of catheter failure due to high pressures created during the procedure. In one aspect, the catheter provided herein is designed to have a relatively higher radial and tensile strength at the proximal segment 4 than at the distal end 6. Accordingly, the catheter disclosed herein is capable of withstanding higher pressures at the proximal section of the shaft than at the distal segment of the shaft. In one aspect, the peak pressure level during fluid injection of up to between about 200-300 psi occurs at the most proximal point of the catheter shaft 3, which can be configured to have tensile material characteristics capable of withstanding the higher pressures without bursting or otherwise materially failing.

In one aspect, the pressure created by fluid injections drops as fluid travels distally down the shaft and approaches systemic pressure as the fluid enters the target vessel. In this aspect, and because of this decreasing pressure gradient, the distal segment of the PICC line can be configured to not have the same burst pressure properties as the proximal segment, where the pressure level is higher. Accordingly, in one aspect, the distal segment of the PICC shaft can be configured to retain its structural integrity during injections even though it has reduced tensile and pressure capabilities.

In addition to the flexural and tensile characteristics of the PICC line described herein, optionally, the catheter can be configured to provide for enhanced visibility of the distal segment 6 under x-ray or fluoroscopic imaging. It is contemplated that the enhanced visibility can be achieved by increasing the radiopaque filler level relative to the polymer at a selected portion of the catheter, such as, for example and without limitation, at least a portion of the distal segment 6 of the catheter.

In one aspect, radiopaque filler materials, usually in the form of a fine powder can be conventionally added to the polymer to increase overall density of the mixture. The increased density serves to block or impede x-ray penetration, thus providing a visual contrast from surrounding tissue and unfilled polymer material. Numerous radiopaque filler materials well known in the art can be used to increase visibility including barium sulfate, tungsten and bismuth salts, including, but not limited to, bismuth subcarbonate, bismuth trioxide, and bismuth oxychloride. Using these radiopaque fillers, physicians can easily visualize the distal segment under x-ray to confirm correct placement within the superior vena cava 13 as shown in FIG. 2.

Turning now to the method of manufacturing the PICC line disclosed herein, several different methods can be used to achieve the varying flexural and strength characteristics described above. Stiffness and tensile strength characteristics are a function of the amount of radiopaque filler as well as the selected durometer of the polymer resin. In one aspect of the invention, the shaft tubing may be extruded using differing durometer resins and differing filler ratios within a single extrusion process. Specifically, the shaft tubing may be extruded using a Total Intermittent Extruded (TIE) process well known in the art and described by Daneneau in U.S. Pat. No. 4,888,146, incorporated herein by reference. In that TIE process, two or more different durometer polymer resins are mixed with varying levels of radiopaque filler and then extruded.

Varying only the levels of filler material does not adequately achieve the desired characteristics of a venous access catheter. When only the filler is varied, although the distal end of the catheter is more radiopaque, it is also less flexible at the distal segment due to the increased level of filler. In one aspect, when the durometer by itself is varied, the resulting shaft has the desired flexibility characteristics, but is not sufficiently visible under x-ray. In one exemplary method of the present invention, the shaft 3 has a first segment 4 of higher durometer resin and less filler, a second segment 5 of mixed durometer resin with a higher ratio of filler, and a distal segment 6 of lower durometer resin with the highest level of filler. With this novel method of varying both the durometer and level of filler throughout the extrusion process, a catheter shaft meeting all the requirements of a PICC line can be produced.

In one exemplary aspect, using the TIE process, the first polymer is supplied by a first extrusion device (not shown) for the distal tube segment 6. A second polymer is supplied by the second extrusion device (not shown) for the proximal segment 4. At the transition tube segment 5, the first polymer flow is shut off while the second polymer flow is opened, resulting in a transition tube segment 5 containing a varying mixture of the first and second polymer materials. Using the TIE process described above produces extruded tubing with varying physical characteristics based on the polymer resin and filler mix.

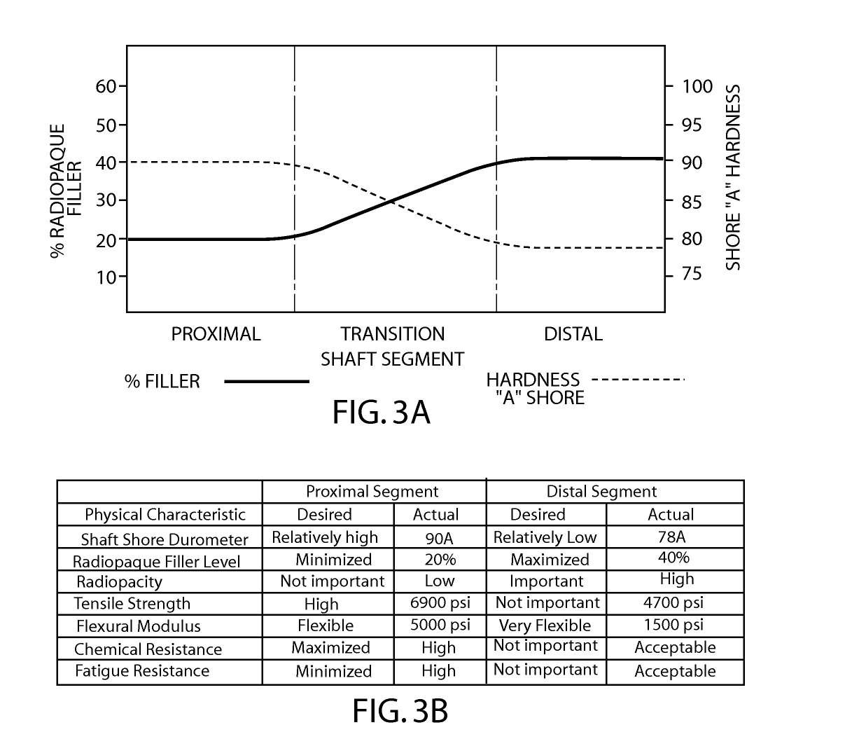

In one aspect, FIG. 3A depicts the varying durometer and radiopaque filler along the length of the catheter shaft after extrusion as described above. In one aspect, as illustrated in FIG. 3A, the percentage of radiopaque filler by weight can range from approximately 10% at the proximal end of the catheter shaft to approximately 50% at the distal end of the catheter shaft. In yet another aspect, the hardness or durometer of the catheter shaft can range from approximately 100 Shore A or 55 Shore D at the proximal end of the catheter shaft to approximately 85 Shore A or 40 Shore D at the distal end of the catheter shaft. Polymer hardness, measured by ASTM D 2240 durometer hardness, can be independent of the percentage of radiopaque filler loading.

In one aspect, as can be seen in FIGS. 3A and 3B, at the proximal segment 4 of the tubing, the radiopaque filler level is approximately 20% radiopaque filler (solid line) and 80% Thermoplastic Polyurethane (TPU) by weight. In one aspect, the filler level increases along the transition tube segment 5 until it reaches approximately 40% filler by weight to approximately 60% TPU by weight at the distal segment 6. Similarly, in another aspect, the TPU durometer, measured in Shore A hardness, decreases from about 55 D at the proximal end to about 95 A at the distal end. In one aspect, as shown in FIG. 3A, the transition segment of the extruded tubing contains a varying degree of both radiopaque filler and TPU durometer.

In one aspect, physical test data on the varying characteristic tubing is illustrated in FIG. 3B. In one aspect, at the proximal end of the catheter, the tensile strength of the shaft is substantially high to provide the necessary strength and durability to the exposed segment of the catheter. In another aspect, the higher durometer polymer combined with a lesser amount of radiopaque filler provides the increased strength and durability characteristics of the proximal segment, as evidenced by the increased tensile strength measurements. Similarly, the chemical and fatigue resistance levels of the proximal portion of the catheter shaft are higher than at the distal segment.

Although the example above utilizes 20% radiopaque filler at the proximal segment of the catheter, in one aspect, the percentage of radiopaque filler by weight used can depend on the density of the filler as well as the specific polymer used. Accordingly, in one exemplary aspect, the proximal segment can contain a range of approximately 0% to approximately 30% radiopaque filler by weight. In another exemplary aspect, the distal segment filler ratio can range from approximately 30% to approximately 50% radiopaque filler by weight. In another exemplary aspect, the percentage radiopaque filler by weight of the proximal segment can be about between about 30 to 80%, between about 40 to 90%, or between about 50 to 100% less than the percentage radiopaque filler by weight of the distal segment.

Similarly, the durometer of the combined polymer and filler material will depend on the specific polymer used. In one exemplary aspect, the durometer of the proximal shaft segment 4 can range from between about 70 Shore D to about 45 Shore D, from between about 65 Shore D to about 50 Shore D, and preferably between about 60 Shore D to about 50 Shore D. In one exemplary aspect, and as shown in FIG. 3A, the durometer of the distal shaft segment can be about 55 Shore D. In another exemplary aspect, the durometer of the distal shaft segment 6 can range from between about 80 Shore A to about 60 Shore D, from between about 85 Shore A to about 57 Shore D, and preferably between about 90 Shore A to about 100 Shore A. In one exemplary aspect, and as shown in FIG. 3A, the durometer of the distal shaft segment can be about 95 Shore A.

In one aspect, the flexural modulus is a measurement of the relative stiffness of an object under applied stress and is measured in pounds per square inch required to bend the object. The higher the flexural modulus measurement, the higher the stiffness. As shown in FIG. 3B, the proximal portion of the catheter shaft has a higher flexural modulus psi and is accordingly stiffer than the distal segment of the shaft. In one aspect, the proximal segment can have a flexural modulus of approximately 6900 psi, and the distal segment can have a flexural modulus of approximately 6000 psi. In one aspect, the radiopacity of the distal end of the catheter shaft, with its higher level of filler, results in a shaft that is more visible under x-ray or fluoroscopy at the distal segment. In one aspect, the increased stiffness created by the higher radiopacity filler load at the distal end is offset by the lower durometer resin, resulting in a distal segment that is both highly visible under x-ray and is flexible and atraumatic to the patient.

Thus, in one aspect, by varying the durometer and the percentage by weight of the radiopaque filler along the shaft length, optimal characteristics of a venous access catheter can be achieved. In one aspect, at the proximal end of the catheter shaft, where the catheter is subject to increased manipulation and exposure to chemicals, the device is fatigue and chemical resistant as well as having increased overall strength as measured by tensile strength. In addition, the increased strength at the proximal end allows for the safe use of power injections with their relatively high-pressure levels. At the distal end, the shaft has enhanced visibility under image guidance as well as a softer, more flexible atraumatic shaft.

In one exemplary aspect, the single extrusion process also ensures a strong transition segment which is less subject to failure under high pressure or tensile force than other welded or bonded transition segments. Accordingly, the absence of welded or bonded points along the catheter shaft will increase durability during insertion, withdrawal and CT injections.

In other aspect, other methods of creating a variable characteristic PICC line are also possible. For example, the TIE extrusion method previously described can be adjusted to create a transition segment that is longer or shorter relative to the distal and proximal segments of the catheter shaft. Specifically, by controlling the speed at which the two base polymers are switched during the extrusion process, the length of the transition segment can be varied. Slowing down the switch over rate from the first polymer mix to a second polymer mix will result in a longer transition segment. In one aspect, the switch over rate can be adjusted such that the majority of the shaft consists of the transition segment, thus creating a continuously variable characteristic catheter shaft. Alternatively, increasing the speed at which the conversion from one polymer mix to the other takes place will create a shaft with a relatively short transition segment.

Another method of extrusion, or means for seamlessly positioning two catheter tubes such that they form one monolithic tub, commonly known in the art as co-extrusion, can also be used to create a variable characteristic venous access catheter described herein. In this aspect, two separate extruders can be utilized to create a single tube with two different material layers. The cross-sectional wall thickness of each layer can then be varied along the length of the shaft. As an example, the outer layer may be extruded using the lower durometer polymer, higher radiopaque filler mixture and the inner layer can be extruded using the higher durometer polymer, lower radiopaque filler mixture. In another embodiment, the outer layer may be extruded using a lower amount of radiopaque filler mixture compared to the inner layer. In one aspect, the outer tubing wall thickness can transition from a smaller percentage of the overall tubing wall cross-section to a larger percentage of the overall tubing wall as it approaches the distal end. Conversely, in one aspect, the inner tubing wall thickness can transition from a larger to a smaller percentage of the overall tubing wall as it approaches the distal end of the shaft. In one aspect, the resulting single tube can consist of substantially entirely the outer layer material at the distal end of the catheter, transitioning to substantially entirely the inner layer material at the proximal end of the catheter.

In one exemplary aspect, the distal segment of the catheter shaft can consist of approximately 90% outer layer with its high radiopacity and relatively low durometer and 10% inner layer, although a range of 75% to approximately 95% outer layer is possible. In another exemplary aspect, at the proximal segment, the catheter shaft can consist of approximately 90% inner layer with its low radiopacity and higher durometer and 10% outer layer. In one aspect, a range of between approximately 75% and approximately 95% inner layer for the proximal segment can be acceptable. Although in the example above, the outer layer can consist of the higher filler, lower durometer material, it is possible to reverse this approach and use the higher filler, lower durometer polymer mixture as the inner layer instead. In one exemplary aspect, with either method, varying the thickness of each layer of the tubing along the length of the shaft can provide a continually varying durometer, strength and radiopacity shaft of an optimal venous access catheter.

One type of co-extrusion process that can be used to produce the catheter shaft 3 is a co-extrusion process that involves a Carbothane® thermoplastic polyurethane (TPU) material (Lubrizol Advanced Materials, Inc., Cleveland, Ohio). The co-extrusion process described herein can be used to produce single, dual, and triple lumen catheters. In one exemplary embodiment the catheter shaft can be a 5 Fr dual lumen catheter shaft.

Carbothane TPUs or carbonate-based urethanes, are a family of medical-grade polycarbonate-based aliphatic polyurethanes. Polyurethanes are desirable because of their high mechanical properties, commercial availability, purity and consistency, non-immunogenicity, ease of fabrication and transportation, sterilizabiltiy, and non-toxicity. Further, polyurethanes are non-teratogenic, non-carcinogenic, and non-mutagenic. Carbothane® TPU materials are advantageous because they do not yellow over time and they have a higher relative degree of biostability compared to ethers and esters. Carbothane® materials are typically available over a wide range of durometers, colors, and radiopacifiers. Carbothane® TPUs also have superior oxidative and hydrolytic stability compared to typical TPU polyethers and polyesters and better solvent interaction than polyethers. Carbothane® TPUs feature better color fastness and chemical resistance compared to aliphatic ether-based urethanes and esters. Carbonate-based urethanes have improved softening properties when exposed to body temperatures and fluids, compared to aliphatic ether-based urethanes. Carbonate-based urethanes are also strong, flexible, alcohol-resistant materials that have enhanced biocompatibility, durability, and can allow for increased ease of care. For at least these reasons, the catheter shaft 3 produced by this co-extrusion process is an improved catheter shaft capable of enhanced performance compared to other catheters.

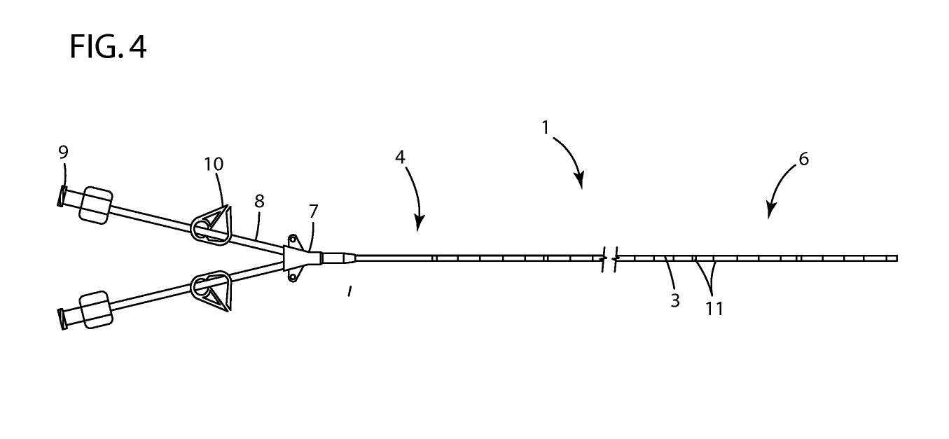

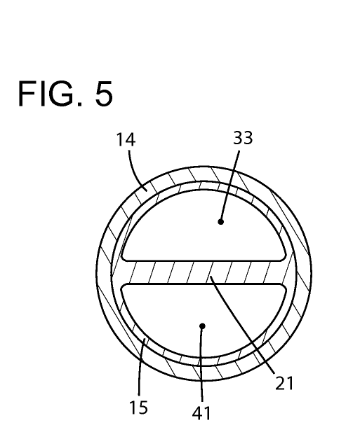

Referring to FIGS. 4 and 5, a catheter shaft produced by the Carbothane® based co-extrusion process described herein is illustrated. This catheter shaft 3 can have two or more tubular members or layers. This co-extrusion process is a means for seamlessly positioning an outer tubular member and an inner tubular member in a coaxial relationship to each other such that the outer tubular member and the inner tubular member together form a monolithic tube. In one aspect, the catheter shaft can have an outer tubular member 14 and an inner tubular member 15, as illustrated in FIG. 5. The outer tubular member 14 can be positioned in a coaxially surrounding relationship to the inner tubular member 15. The outer tubular member 14 has an outer surface and an inner surface and a first wall thickness. The inner tubular member 15 has an outer surface and an inner surface and a second wall thickness. At least a portion of the inner surface of the outer tubular member 14 is positioned such that it is in contacting relationship with at least a portion of the outer surface of the inner tubular member 15. In one exemplary embodiment, the entire inner surface of the outer tubular member 14 is in contacting relationship with the entire outer surface of the inner tubular member 15 along a longitudinal length of the catheter shaft. The inner tubular member 15 comprises a septum 21 that separates inflow and outflow lumens 33, 41. One of ordinary skill in the art will recognize that the catheter shaft can comprise one, two, or more lumens. The septum 21 extends along the longitudinal length of the catheter shaft 3 and is comprised of the same material as the inner tubular member 15. The septum 21 and the inner tubular member 15 can form a unitary inner tubular member 15. In another aspect, the septum 21 can be at least partially comprised of the material of the inner tubular member 15. In yet another aspect, the septum 21 can be at least partially comprised of the material of the outer tubular member 14.

The catheter shaft has a total wall thickness that is comprised of at least a wall thickness of the outer tubular member and a wall thickness of the inner tubular member. The wall thicknesses of either or both of the inner tubular member 15 and the outer tubular member 14 can be varied. In one aspect, the wall thicknesses can be varied continuously along the length of the catheter shaft. In another embodiment, the wall thickness along the length of the catheter shaft can remain constant. The wall thickness of either or both of the outer tubular member and the inner tubular member can continuously decrease along the length of the catheter shaft from the proximal end to the distal end of the shaft. In one aspect, the inner tubular member 15 wall thickness can vary from about 25% to about 75% of the total wall thickness, while the outer tubular member 14 wall thickness comprises the remainder of the total wall thickness. The wall thickness of the outer tubular member 14 can range from about 25% to about 75% or the total wall thickness, while the inner tubular member 14 comprises the remainder of the total wall thickness. In one aspect, the wall thickness of either or both of the outer tubular member 14 and the inner tubular member 15 can range from about 30% to about 70% of the total thickness of the catheter shaft.

The inner and outer tubular members 15, 14 can have the same or different properties that allow for enhanced performance of the catheter shaft 3 after the catheter 1 is inserted into a patient's body and during treatment of a patient. In one exemplary embodiment, the inner tubular member 15 and the outer tubular member 14 can be comprised of different materials. In another aspect, the inner tubular member 15 and the outer tubular member 14 can be comprised of the same materials. For example, the inner and outer tubular members 15, 14 can be comprised of carbonate-based urethanes. In another embodiment, inner and outer tubular members 15, 14 can be comprised of a Tecoflex® material. Tecoflex® is a family of medical-grade aliphatic polyether polyurethanes. They do not yellow over time and are available over a wide range of durometers, colors, and radiopaque filler materials, including barium sulfate, bismuth salts, and tungsten.

In another embodiment, inner and outer tubular members 15, 14 can be comprised of materials having the same durometer. In another aspect, the inner and outer tubular members 15, 14 can be comprised of materials having different durometers. In one embodiment, the outer tubular member 14 can be comprised of a material that can be substantially clear or substantially transparent. All or a portion of the outer tubular member can be clear. In another embodiment, the inner tubular member 15 can be substantially clear or transparent. All or a portion of the inner tubular member 15 can be substantially clear. The inner tubular member 15 can be colored. In one example, the inner tubular member 15 can be, but is not limited to, a blue color. In yet another embodiment, both the inner tubular member 15 and the outer tubular member 14 can be substantially clear or transparent.

The inner tubular member 15 can have a first strength, a first durometer, a first modulus, and a first tackiness. The outer tubular member 14 can have a second strength, a second durometer, a second modulus, and a second tackiness. Tacky means sticky or slightly adhesive or gummy to the touch. Tackiness means how sticky a material is and is indirectly proportional to the durometer of a material. The inner tubular member 15 is less tacky or has a lower tackiness compared to the tackiness of the outer tubular member 14. The first strength, first modulus, and first durometer of the inner tubular member 15 can each be greater than the second strength, the second modulus, and the second durometer of the outer tubular member 14. Modulus is a measure of stress over strain or overall stiffness of a material. Generally, the softer the material, the lower the modulus and the strength. The durometer of the inner tubular member 15 can range from about 55 D to about 95 A. In one aspect, the durometer of the inner tubular member 15 can vary from about 55 D to about 85 A. Durometer is one of several measures of the hardness of a material, such as a polymer, elastomer, or a rubber. Hardness can be defined as a material's resistance to permanent indentation. The term durometer also refers to a measurement device used to measure the durometer of a material. There are several scales of durometers that can be used for materials having different properties. They are the ASTM D2240 type A for softer plastics and the type D scales for harder plastics. The ASTM D2240-00 testing standard calls for a total of 12 scales, depending on the intended use. Each scale results in a value between 0 and 100, with higher values indicating a harder material.

The durometer of the outer tubular member 14 can be from about 75 A to about 95 A. This combination of two materials, namely, the outer softer tubular member with a lower durometer and a harder inner tubular member with a higher durometer, compared to the outer tubular member allows the catheter shaft 3 to have an improved, increased strength without a significant increase in overall catheter stiffness, while maintaining the desired flexibility along the entire length of the catheter shaft. This allows the catheter shaft to accommodate high pressures during injection, yet allows for enhanced patient comfort.

While the higher durometer inner tubular member 15 has a higher strength compared to the outer tubular member 14, the outer tubular member 14 can have improved flexibility to withstand higher pressures which allows the catheter to accommodate higher flow rates. The flexibility of the outer tubular member 14 can be greater than the inner tubular member 15. The higher durometer of the inner tubular member 15 can provide improved chemical resistance. This configuration can also provide a lower coefficient of friction than a material with a lower durometer, strength, and modulus. This allows the catheter to glide through tissue with increased ease. This configuration provides the catheter shaft with a lower overall modulus compared to a catheter having a single higher durometer. The catheter shaft is less tacky than if the catheter shaft was comprised entirely from a single lower durometer material. This configuration is also advantageous because it can result in reduced force required by a practitioner to remove guidewires from a lumen of the catheter shaft.

To further aid in maneuverability of a guidewire, at least a portion of the inner tubular member 15 may comprise a coating, such as, but not limited to, a lubricant. The lubricant may be dispersed and blended throughout the material of the inner tubular member 15. In another aspect, the lubricant can be coated onto at least a portion of the inner surface of the inner tubular member 15. The lubricant coating can allow the catheter shaft to have a decreased coefficient of friction, compared to a catheter lumen without a lubricant. The inner tubular member polymer and the lubricant composition can be coextruded so that the lubricant composition is laminated onto the inner surface of the inner tubular member.

The addition of a lubricant can reduce the force necessary to insert or remove a guidewire from a lumen of the catheter. The lubricant may be compatible with urethane, and may be, but is not limited to, a wax or lubricious polymer, for example, PTFE. The outer tubular member 14 may have no lubricant. The absence of lubricant on the outer surface of the outer tubular member 14 can help prevent erosion of any printing or other indexing marks on the outer surface of the outer tubular member 15.

The Carbothane®-based materials of the inner and outer tubular members, described above, can be comprised of various ratios of three components, such as, but not limited to, Macroglycol/diol, chain extender/(diol), and isocyanate. The ratios between these three components can be varied, depending on the desired softness or hardness of the inner or outer tubular members 15, 14. Macroglycol/diol comprises a soft segment of the tubular members 14, 15. The soft segment can be a high-molecular-weight macroglycol. The chain extender, and isocyanate comprise the hard segment of the tubular members 14, 15. The hard segment is formed by the reaction between a diisocyanate with a low molecular weight diol, such as, but not limited to, ethylene glycol or 1,4-butanediol (BDO). The chain extender can be categorized into two general classes of aromatic diol and diamines and the corresponding aliphatic diols and diamines. In one aspect, the chain extender can be a low molecular weight organic diol. In general, aliphatic chain extenders yield softer materials than aromatic chain extenders. Chain extenders can be used to extend the length of the hard segment and increase the hydrogen-bond density and the molecular weight of the polyurethane. The ratio of soft to hard elements of the materials that comprise the inner tubular member 15 and the outer tubular member 14 determines the durometer of each of the members 14, 15, in addition to other properties, such as, but not limited to the tensile strength, elongation and tensile modulus. Although the inner tubular member 15 and the outer tubular member 14 can be comprised of materials having different durometers, as described above, in another exemplary embodiment, the inner tubular member 15 and the outer tubular member 14 can be comprised of materials having the same durometers. In one exemplary embodiment, either or both of the inner tubular member 15 and the outer tubular member 14 can be an aliphatic carbonate urethane.

In one exemplary embodiment, either or both of the outer tubular member 14 and the inner tubular member 15 can comprise at least one radiopaque filler. The radiopaque filler can range from about 0% to about 40% by weight in either or both of the inner tubular member 15 and the outer tubular member 14. In one aspect, the filler material can be evenly dispersed throughout the inner tubular member 15 and the outer tubular member 14. In one aspect, one of skill in the art would appreciate that it is contemplated that the radiopaque filler material can be formed in any desired geometric shape on any portion of the catheter tube.

The properties of the inner and outer tubular members 15, 14 can be modified by varying the amount of filler in one or both of the inner and outer tubular members. In one exemplary embodiment, the inner tubular member 15 can have a first amount of radiopaque filler by percentage weight, and the outer tubular member 14 can have a second amount of radiopaque filler by percentage weight. The first amount of radiopaque filler can be greater than the second amount of radiopaque filler. In one aspect, the outer tubular member 14 may optionally contain 0% by weight filler material. The advantage of having an outer tubular member 14 with 0% radiopaque filler is that this decreases the chance that pathways can form within the polymer material of the outer tubular member 14. This could cause the polymer material to be discontinuous. This could also further lead to decreased chemical resistance of the outer tubular member 14. Cracks could form upon exposure to chemicals and mechanical stresses. Chemicals and/or other substances, such as blood, could leach into the material of the outer tubular member 14 which could cause the polymer material of the outer tubular member 14 to break down. This could further lead to oxidation of poly(ether)urethane surfaces when implanted, macrophage adhesion and activation, induced or residual mechanical stress, and the breakage of susceptible ether linkages. The use of a polycarbonate urethane material in the outer tubular member 14, combined with a higher amount of radiopaque filler in the inner tubular member 15 compared to the outer tubular member 14 provides a catheter shaft 3 having an increased durometer, strength, and modulus. This combination also provides for a lower coefficient of friction than a material with a lower amount of radiopaque filler or no radiopaque filler, resulting in a lower durometer, strength, and modulus. In yet another embodiment, the first amount of radiopaque filler can be less than the second amount of radiopaque filler. This catheter design provides a catheter shaft with a lower overall modulus compared to a catheter shaft having a single radiopaque filler material in both of the inner and outer tubular members 15, 14.

The co-extrusion process described herein can be used herein to modify the properties of the inner tubular members and the outer tubular members, either individually, or in combination, as described above, to produce a catheter shaft. This catheter shaft can have improved retained flexibility and patient comfort during insertion and use in a patient, while also providing an improved resistance to high pressure flow rates during CT injections, such as those that occur during insertion and use of a PICC.

The co-extrusion process described herein provides a catheter shaft that is capable of improved performance during insertion and use in a patient, including improved maneuverability and/or ease of guidewire insertion, advancement, and withdrawal during use and the capability of accommodating high pressure injections.

Various omissions, modifications, substitutions and changes in the forms and details of the device illustrated and in its operation can be made by those skilled in the art without departing in any way from the spirit of the present invention. Accordingly, the scope of the invention is not limited to the foregoing specification, but instead is given by the appended claims along with their full range of equivalents.

What is claimed is:

1. A catheter shaft, comprising:
a monolithic tube comprising:
an outer tubular member having an inner surface and an outer surface, wherein the outer tubular member comprises a polymer material of a first durometer and a first amount of a radiopaque filler; and
an inner tubular member comprising an inner surface and an outer surface, wherein the inner tubular member comprises a polymer material of a second durometer and a second amount of the radiopaque filler, wherein the second durometer is greater than the first durometer, and wherein the outer tubular member is defined in a coaxially surrounding relationship to the inner tubular member, wherein the outer tubular member and the inner tubular member define a total catheter wall thickness.

2. The catheter shaft of claim 1, wherein the outer tubular member has a first strength, wherein inner tubular member has a second strength, and wherein the second strength is greater than the first strength.

3. The catheter shaft of claim 1, wherein the outer tubular member is substantially transparent.

4. The catheter shaft of claim 1, wherein the outer tubular member has a first modulus, and wherein the inner tubular member has a second modulus, and wherein the second modulus is greater than the first modulus.

5. The catheter shaft of claim 1, wherein at least a portion of the inner surface of the outer tubular member is positioned in contacting relationship to at least a portion of the outer surface of the inner tubular member.

6. The catheter shaft of claim 1, wherein the percentage by weight of the radiopaque filler comprised therein the inner tubular member is greater than the percentage by weight of radiopaque filler material in the outer layer.

7. The catheter shaft of claim 1, wherein the filler material in the inner tubular member is from about 0% to about 40% by weight.

8. The catheter shaft of claim 1, wherein the filler material in the outer tubular member is from about 0% to about 40% by weight.

9. The catheter shaft of claim 8, wherein the filler material in the outer tubular member is about 0% by weight.

10. The catheter shaft of claim 1, wherein the total catheter wall thickness varies over the length of the catheter shaft.

11. The catheter shaft of claim 1, wherein the outer tubular member has a wall thickness, and wherein the inner tubular member has a wall thickness.

12. The catheter shaft of claim 11, wherein the wall thickness of the inner tubular member is greater than the wall thickness of the outer tubular member.

13. The catheter shaft of claim 11, wherein the wall thickness of the outer tubular member is greater than the wall thickness of the inner tubular member.

14. The catheter shaft of claim 11, wherein the thickness of the outer tubular member comprises from about 25% to about 75% of the total wall thickness.

15. The catheter shaft of claim 11, wherein the thickness of the inner tubular member comprises from about 25% to about 75% of the total wall thickness.

16. The catheter shaft of claim 11, wherein the wall thickness of the outer tubular member varies over the length of the catheter shaft.

17. The catheter shaft of claim 11, wherein the wall thickness of the inner tubular member varies over the length of the catheter shaft.

18. The catheter shaft of claim 1, wherein the monolithic tube defines one or more lumens.

19. The catheter shaft of claim 1, wherein the catheter defines two lumens.

20. The catheter shaft of claim 1, further comprising a hub component attached to the proximal tube segment and configured to remain outside of a patient body.

21. The catheter shaft of claim 1, wherein the outer tubular member is more flexible than the inner tubular member.

22. The catheter shaft of claim 1, wherein the flexibility of the catheter shaft varies along the length of the catheter shaft.

23. A catheter shaft, comprising:
an outer tubular member having an inner surface and an outer surface, wherein the outer tubular member comprises a polymer material of a first durometer and a first amount of a radiopaque filler; and
an inner tubular member comprising an inner surface and an outer surface, wherein the inner tubular member comprises a polymer material of a second durometer and a second amount of the radiopaque filler, wherein the second durometer is greater than the first durometer, and wherein the outer tubular member is defined in a coaxially surrounding relationship to the inner tubular member, wherein the outer tubular member and the inner tubular member define a total catheter wall thickness; and
a means for seamlessly positioning the outer tubular member and the inner tubular member in a coaxial relationship to each other such that the outer tubular member and the inner tubular member together form a monolithic tube.

24. An integral venous access catheter shaft comprising:
a monolithic tube comprising:
a proximal tube segment of a first polymer material comprising less than 30% by weight radiopaque filler and having a first durometer between about 70 Shore D to about 45 Shore D, wherein the proximal tube segment has a proximal tube segment burst strength;
a distal tube segment of a second polymer material comprising between about 30% and 50% by weight radiopaque filler and having a second durometer between about 80 Shore A and 60 Shore D, wherein the second durometer value is less than the first durometer value, wherein the burst strength of the distal tube segment is less than the proximal tube segment burst strength, and wherein the percentage radiopaque filler by weight of the proximal tube segment is between about 30 to 80% less than the percentage radiopaque filler by weight of the distal tube segment; and
a transition tube segment seamlessly interposed between the proximal and the distal tube segments to form the monolithic tube, the transition tube segment having a varying mixture of the first and second polymer materials, wherein the ratio of the first polymer material to the second polymer material decreases from a proximal end to a distal end of the transition tube segment, and wherein the percentage by weight of the filler increases from the proximal end to the distal end of the transition segment.
